(19) United States
(12) Patent Application Publication
(10) Pub. No.: US 2002/0108869 A1
(43) Pub. Date: Aug. 15, 2002

(54) Device and technique for multiple channel patch clamp recordings

(76) Inventor: Alex Savtchenko, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/779,955

(22) Filed: Feb. 9, 2001

(51) Int. Cl. C12N 13/00

(52) U.S. Cl. 205/777.5; 435/285.2

(57) ABSTRACT

A device to facilitate electrophysiological measurements of a biological material comprises a plate having a plurality of wells that each have an end. At least some of the wells have a hole formed in the end, and the holes are configured to receive an individual cell such that a high resistance seal is formed between the cell and the end. A chamber is disposed adjacent the plate and is in fluid communication with each of the holes. A common electrode is disposed in the chamber, and a plurality of well electrodes are configured to be positioned within the wells. In this way, a voltage gradient may be created across cell membranes of cells that are positioned within the holes so that electrophysiological measurements of the cells may be taken.

### BACKGROUND OF THE INVENTION

1. Field of the Invention

This invention relates generally to electrophysiological evaluations of biological materials. More specifically, the invention is related to devices and techniques for evaluating ion channels in cell membranes in a high throughput manner. The invention is further related to techniques for creating a high resistance seal between a cell and the wall of a recording probe to facilitate electrophysiological measurements.

2. Background Art

The patch clamp method is a technique that enables the recording of currents flowing through the ion channels located on the surface of a cell membrane. In brief, the patch clamp method uses the unique property of the cellular membrane to form a tight seal contact, known in the art as a “Giga-seal”, between the membrane itself and the wall of the recording probe. Such a seal has a high resistance that facilitates measurement of currents flowing through the cell membrane with high precision. Such measurements may then be used to evaluate the effectiveness of a given drug.

A detailed summary of the development of the patch clamp technique is described in, for example, PCT publication nos. WO 96/13721 and WO 99/66329, the complete disclosures of which are herein incorporated by reference. In its existing form, the patch clamp method is a low throughput assay for ion channel drug discovery. Formation of the high resistance seal is tedious and requires special training and expensive equipment. An experienced electrophysiologist now can screen only about 5 to 20 compounds a day using traditional patch clamp techniques. This causes a major bottleneck in the screening process since ion channel ligands identified in other types of assays often need to be confirmed in a patch clamp assay.

Other existing methods of electrophysiological recordings include the use of a two microelectrode voltage clamp, extracellular recordings, and the “U-tube” method. Although less demanding in terms of equipment and personnel training, these techniques do not satisfy the current requirements for high throughput screening.

Alternative methods of recording ion channel activity, such as optical methods of recording the voltage change across the cell membrane, have much higher throughput. However, these methods lack the precision and the information content of the electrophysiological methods for screening purposes and cannot provide the amount of information one can gain from electrophysiological recordings.

Hence, this invention is related to other techniques and equipment for evaluating the electrophysiological attributes of a biological material. Such equipment and techniques are particularly suited to significantly increase the throughput of physiological measurements, including patch clamp type experiments.

### SUMMARY OF THE INVENTION

In one embodiment, the invention provides a novel way to create an electrically resistive or tight seal between a cell membrane and the wall of a recording probe to facilitate the measurement of currents flowing through the cell membrane. Such a seal may be used with existing patch clamp type experiments, and may also be used to facilitate high throughput screening procedures. In another embodiment, the invention provides novel ways to high throughput screen ion channel assays. Such screening techniques may utilize the novel resistive seal, or may utilize Giga-seals used in traditional patch clamp experiments.

In one particular embodiment, the invention provides a device to facilitate electrophysiological measurements of a biological material. The device comprises at least one well having an end and a side wall in the end that defines an opening. A glue-like substance is disposed on the side wall of the opening and is used to create a high resistance seal between a cell membrane and the side wall. A first electrode is provided that may be positioned in the well along with a second electrode that may be positioned outside the well. In this way, the electrodes are separated by a dielectric so that a voltage gradient may be produced across the membrane of a cell that is positioned within the opening to permit electrophysiological measurements of the cell membrane to be taken and recorded.

Use of the glue-like substance permits a highly resistive seal to be made so that high precision measurements may be made without the need for a traditional Giga-seal. For example, the glue may be configured to create a high resistance seal having a leakage resistance of about 600 mega-ohms to about 1.1 giga-ohms. Such a resistance can be less than the resistance of a traditional Giga-seal (a seal having a leakage resistance that is greater than about one giga-ohm), but still large enough for precise measurements. In one aspect, the glue comprises a silicone base glue. This type of seal also facilitates high throughput screens by enabling multiple seals to be created when simultaneously evaluating multiple cells using electrophysiological techniques.

In another embodiment, the invention provides a device to facilitate electrophysiological measurements of a biological material that comprises a plate having a plurality of wells that each have an end. At least some of the wells have a hole that is formed in the end for receiving an individual cell. The hole is configured such that a high resistance seal is formed between the cell and the end when the cell is forced into the hole. A chamber is disposed adjacent the plate and is used to hold an electrically conductive solution. A common electrode is disposed in the chamber, and a plurality of well electrodes are provided that may be positioned within the wells to create a voltage gradient across cell membranes of the cells that are positioned within the holes. In this way, electrophysiological measurements of multiple cells may be taken at the same time.

In one aspect, each hole is tapered, either toward or away from the end. In another aspect, the narrowest dimension of each hole is in the range from about 1 μm to about 5 μm. The seal that is created in each hole may be formed by using a glue that is deposited in the cell. Alternatively, a traditional Giga-seal may be created by using a pressure differential between the well and the chamber.

In another aspect, a multi-channel liquid dispensing system is provided that has a plurality of dispensers that are configured to place the cells in solution into the wells. In this way, each well may rapidly be provided with a cell. Conveniently, the well electrodes may be coupled to the dispensers.

In a further aspect, a vacuum source is coupled to the chamber to produce a vacuum within the chamber. Such a vacuum facilitates the deposition of the cell within the hole to create the resistive seal. Alternatively, positive pressure may be provided from each dispenser and into the well. Conveniently, each dispenser may include a seal member to form a seal with the well such that positive pressure may be supplied to each well.

In still another aspect, electronics are provided to measure voltage and/or current values for each of the wells. A controller may also be provided to control operation of the liquid dispensing system and the electronics. Further, a voltage source is coupled to the common electrode to create the voltage gradient.

In one optional aspect, means are provided for producing a penetrated patch. This may be accomplished, for example, by use of a cutter that is disposed adjacent the plate. The cutter is reciprocatable to severe or produce one or more holes in cells extending below the ends of the wells. Conveniently, the common electrode may be configured to function as the cutter. In this way, the interior of the cell may be placed at the same electrical potential as one of the common electrodes. Alternatively, the bottom of the cell may be perforated using pressure or electrical pulses or by using a Nystatin or other hole forming solution.

The invention further provides a method for evaluating electrical currents flowing through ion channels of the cell. The method utilizes at least one well having an end and a side wall in the end that forms an opening through the end of the well. A glue-like substance is placed on the side wall of the opening and one or more cells are deposited into the opening. The glue is used to create a high resistance seal between the cell and the side wall hole formation. A potential difference is then created across the cell membrane and voltage and/or current measurements are taken and recorded. Hence, such a method produces a high resistance seal (that may be less than a traditional Giga-seal) that is sufficient to make precise electrophysiological measurements.

Advantageously, the glue-like substance may be placed onto side walls of a plurality of wells. In this way, multiple cells may be simultaneously screened by placing them into individual wells where the high resistance seal is produced between each cell and the side wall hole formation of each well. A potential difference may then be created across each cell membrane and appropriate electrophysiological measurements taken and recorded.

In another embodiment, the invention provides a method for evaluating electrical currents flowing through ion channels of a plurality of cells. This method utilizes a plate having a plurality of wells that each have an end. At least some of the wells have a hole formed in the end, and a chamber is disposed below the plate and is filled with an electrolyte solution. A common electrode is also disposed in the chamber. With such configuration, cells are dispensed in a solution into the wells. A pressure differential is applied between the wells and the chamber to collect cells into the holes and to create a high resistance seal between the cells and the ends of the wells. A potential difference is produced between the common electrode and well electrodes that are positioned within each well. Electrophysiological measurements are taken for the cells that are positioned within the holes. In this way, a plurality of cells may be evaluated in parallel to create a high throughput screening system. Alternatively, cells may be deposited into the chamber and then drawn into the holes so that only a small portion of the cells are within the holes. The portions of the cells extending into the chamber may then be penetrated and measurements taken as previously described.

In one aspect, a test is performed to determine whether an appropriate seal has been created between the cells and the ends of the wells. In another aspect, the high resistance seal is a Giga-seal, having a resistance of about one giga-ohm or greater, typically being about one giga-ohm to 1,000 giga-ohms. Alternatively, a glue may be placed into the holes to create the seal between the cells and the ends of the wells. Such a glue may produce a high resistance seal of about 600 mega-ohms to about 1.1 giga-ohms.

### BRIEF DESCRIPTION OF THE DRAWINGS

FIG. 1 is a schematic diagram illustrating a screening technique according to the invention.

FIG. 2 is front perspective view of a high throughput screening device according to the invention.

FIG. 3 is a cross sectional schematic diagram of the screening apparatus of FIG. 2.

FIG. 4 is a front perspective view of a multi-well plate that is used in connection with the screening device of FIG. 3.

FIG. 5 is a schematic diagram of an alternative screening device according to the invention.

FIG. 6 is a more detailed view of a through hole in one of the wells of the screening device of FIG. 5 and further illustrating a common electrode disposed into the well.

FIG. 7 illustrates the screening device of FIG. 6 after a cell has been drawn into the hole.

FIG. 8 illustrates the common electrode that is translated to severe a portion of the cell that is sealed to the through hole.

FIG. 9 illustrates the common electrode when moved back to its home position so that electrophysiological measurements may be taken.

FIG. 10 is a top perspective view of one embodiment of a multi-well plate that may be used in a high throughput screening device according to the invention.

FIG. 11 is a top view of the multi-well plate of FIG. 10.

FIG. 11A is a more detailed view of a well of the multi-well plate of the FIG. 11 taken along section A.

FIG. 11B is a more detailed view of the well of FIG. 11A taken along section B.

FIG. 11C is a cross sectional side view of one of the wells of the multi-well plate of FIG. 11.

FIG. 11D is a more detailed view of a through hole in the well of FIG. 11C taken along section D.

FIG. 11E is a more detailed view of the through hole of FIG. 11D.

FIG. 12 is a graph illustrating voltage and current measurements taken of a cell membrane using the techniques of the invention.

FIG. 13 illustrates an alternative well design according to the invention.

### DESCRIPTION OF THE SPECIFIC EMBODIMENTS

The invention provides devices and methods for enabling the automated screening of ion channel assays and allows the parallel processing of many compounds and many cells at once. The devices and techniques of the invention may utilize a traditional Giga-seal or other high resistance seal contact between a cell and a hole in the well of a multi-well plate. In this way, the invention provides the ability to screen the same compound against multiple targets in the same experiment. For example, the invention may utilize native cell lines with multiple channels expressed to permit the screening of the same drug molecule against multiple target ion channels within the same experiment which increases throughput multiple times. When used as a drug discovery tool, the invention may be used to determine whether drugs are good modulators of ion channels. Diseases associated with the modulation of ion channel function include the cardiovascular area, including hypertension and cardiac arrhythmias, pain (local anesthetic), diabetes, ellipsy, anxiety, and the like. The invention may also be used in the estimation of the ion channel genes present in the human genome (for future targets of drug discovery).

The invention also provides the ability to use the ion channels as “biosensors”. For example, the invention may be used to measure pH changes. Further, some cell lines may be used to evaluate the effect of the same drug onto specific kinase, phosphorylating the test ion channel and the effect of this drug on the channel itself. This gives a readout of the drug effect.

Hence, ion channels may be used as biosensors since ion channels are indicators of the result of drug action onto other molecular targets inside the cells. This includes kinase/phosphatase modulation, which in turn changes the kinetic behavior of certain ion channels and may be recorded with high precision using electrophysiological assays. It may also include proton sensitive channels that are natural pH meters (alternative for micro physiometer).

The electrophysiological information output from a single experiment of the invention can be up to about 25 parameters that are recorded essentially simultaneously. The techniques of the invention also provide the ability to dialyze the cell cytoplasm, thus allowing one to manipulate with the intracellular solution composition, introducing or removing certain ions from the intracellular solution. In this way, a research may dialyze to evaluate one type of channel while excluding other channel types. This permits the optimization of one channel while excluding all others. Further, the electrophysiological methods have high sensitivity, allowing one to record the activity of a single channel molecule. The techniques of the invention also have high temporal resolution (in sub-millisecond range) which is necessary for some ion channel targets, such as fast inactivating Na channels.

In one embodiment, the invention provides specific devices and methods for performing multiple channel patch clamp experiments. One such device may include a multi-well plate with an end that forms the end of each well. Conveniently, the end may be formed of glass, plastic or other dielectric material. A small through hole is formed in the end, and a chamber that is filled with an electrolyte solution is positioned adjacent the plate. The device may further include a vacuum system for creating a vacuum in the chamber and may include appropriate controls for controlling the vacuum. A dispensing device is employed to dispense compounds into each well, and electrodes and electronics are provided to measure the current and voltage for the cell being studied in each well.

One type of multi-well plate that may be used is one having a plastic body with a solid glass bottom. An example of such a plate is model No. 7706-2375, commercially available from Whatman Polyfiltronics. A small conical hole is drilled into the glass bottom of each well using a laser drilling technique. The hole may be provided with an exit diameter in a range of about 1 μm to about 5 μm. The wall may have an angle of taper of about 90° through both dimensions. However, this angle may vary depending upon the type of cell being studied, among other variables.

The multi-well plate may be located above and sealed to a chamber which contains both an electrolytic solution and an electrode that is common to each of the wells. However, it will be appreciated that multiple electrodes could be used within the chamber. The chamber may also have a provision for filling, draining and maintaining a small vacuum to draw cells into each of the wells.

Located above the multi-well plate is a multi-well dispensing device and an electrode for each well. The electrode may conveniently be part of the multi-well dispensing device, or may be separate. For example, the electrodes may be incorporated into the sides of the wells, may be a thin film electrode on the sides or ends of the wells, or the like. Each of the electrodes is connected to electronics designed to measure the current and voltage between each individual well and the common electrode in the chamber, i.e. through the hole in the dielectric material.

Cells in solution are added to each well of the plate, and the plate is attached to a chamber which is filled with the electrolytic solution. A small vacuum may then be drawn to pull a single cell into the tapered hole and form a high resistance seal. Alternatively, positive pressure could be supplied from the top to position the cell within the tapered hole.

The seal formed between the cell and the wall of the tapered hole may be a traditional Giga-seal having a resistance of about one giga-ohm or greater. Alternatively, a glue-like substance may be placed onto the walls of each well. This may be accomplished, for example, by dipping the bottom of the multi-well plate in a reservoir containing the glue-like substance and then removing the excess glue by shaking the plate or by applying a small pressure to one side of the plate. Once dispensed in the well, the cells will form a tight sealed contact with the wall of each well allowing electrophysiological measurements. The glue-like substance may comprise a silicon-based glue, a Vaseline/paraffin-based composition, or the like. Such a glue-like substance is preferably a chemically inert, soft grease-like substance. This allows the cell to stick to the surface of the through hole and form the seal with a leakage resistance of around 600 mega-ohms to about 1.1 giga-ohms. Such a leakage resistance is sufficiently high for whole-cell recordings.

The multi-well plate may conveniently have a standard footprint. For example, the plate may have wells in number of 96, 864, 1564, or the like as is known in the art.

Such a system allows for multiple compounds to be distributed into the wells during the same experiment. Once in the wells, the wells may be sealed, allowing for the application of pressure into each individual well separately as previously described. Conveniently, the dispensing needle of the dispensing device may serve as the measuring electrode for whole-cell recordings. Another advantage of such a feature is that such plates may be manufactured inexpensively and are disposable.

One exemplary procedure for performing a screening experiment is by providing a cell line with expressed target ion channels. Each well is configured to receive a few of these cells, although only one cell per well is needed. The plate is placed onto the chamber having an intracellular solution. The common electrode positioned in the chamber may conveniently be constructed of a metal plate that may be shifted to allow the solution to flow downward from each of the wells. Further, it will be appreciated that more than one common electrode may be used. For example, two or three common electrodes may be used. A slight positive pressure may be applied to each well, or a vacuum may be supplied to permit the cells to plug the through holes, thereby blocking them. Such procedure may take about 1 to 3 minutes to permit the cells to form high resistance seals with the holes in the end of each well. When the appropriate seal has been produced, a voltage of about −70 mV voltage difference is produced between the intracellular electrode (the common electrode that is formed from a metal plate) and each of the needles that are disposed in the well. The metal plate may then be shifted back to perforate the lower portion of the cells which are put through each well by the applied pressure. Alternatively, pressure pulses or a perforation solution may be used to perforate the cells. As another alternative, the cells may be penetrated by electroporation. After perforating the lower portion of the cells, the system is ready to record electrophysiological measurements in a high throughput manner.

Before taking measurements, each well may be tested to determine whether the seal has been formed. If not, the well is labeled as a well having a “bad” seal and will be discarded from subsequent considerations. The plate may be tested multiple times during the experiment to reconfirm the stability of seal formation. Each well may be tested by applying small hyper-polarized pulses to the cell membranes. By excluding the “bad” wells from further consideration, ligands are effectively saved by applying them only to the “successful” wells.

Individual cell voltage and current measurements may then be taken and recorded. The recorded data is stored and evaluated to determine the effectiveness of the compounds being tested. Hence, such a technique permits the use of simple and inexpensive multi-well plates that are constructed of plastic, rather than costly silicon and nitride or glass multi-usage plates as are currently being used. Further, the cells may be evaluated in a high throughput manner, using a traditional Giga-seal or other high resistance seal created using a glue-like substance.

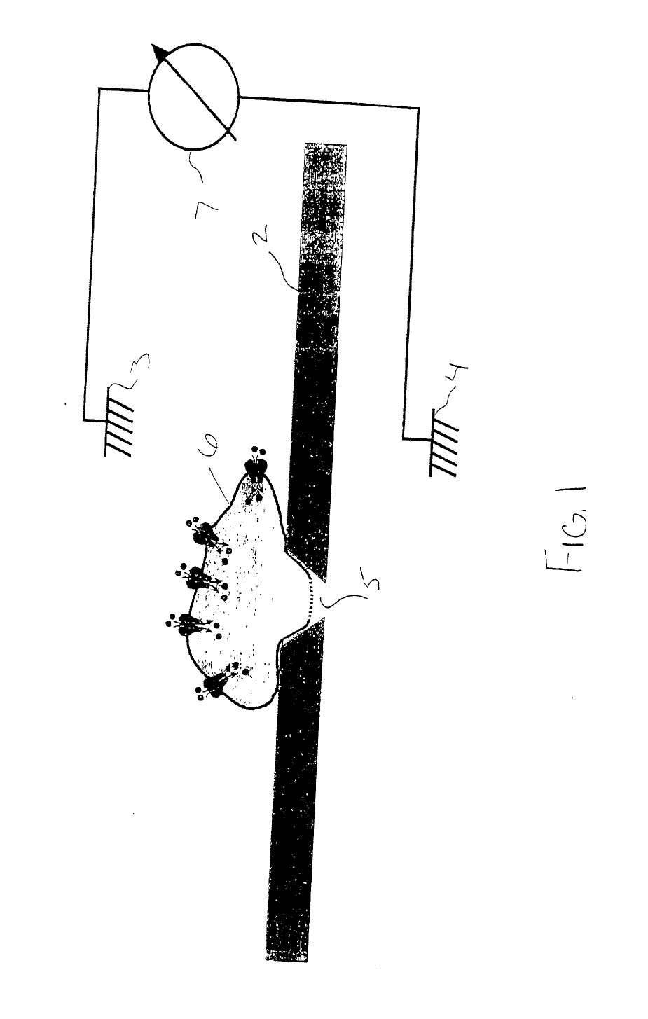

Referring now to FIG. 1, a technique for taking electrophysiological measurements will be described. This technique utilizes a dielectric 2 that is used to separate a pair of electrodes 3 and 4. The dielectric may be of any configuration or shape, and may conveniently be integrated into another structure, e.g. the bottom, side or top end of a well or a chamber. Hence, a primary function of dielectric 2 is to separate electrodes 3 and 4. Dielectric 2 includes a hole 5 for receiving a cell 6. Once one or more pores are created in cell 6, a measuring device 7 may be used to take and record current or voltage values. According to the invention, the dielectric (or multiple dielectrics) includes multiple holes and multiple electrodes so that multiple cells may be evaluated in parallel.

Referring now to FIG. 2, one embodiment of an electrophysiological measuring device 10 will be described. Device 10 is constructed of a housing 12 having a pair of inputs 14 and 16 into which multi-well plates may be inserted. One of the plates may include cells while the other plate holds solutions for transfer to the plate with cells. Positioned above input 14 and 16 are a set of control buttons 18 for controlling operation of device 10. For example, control buttons 18 may be employed to dispense cells into the wells of the multi-well plates, to apply a pressure differential, to create a voltage gradient, to display various measured electrophysiological parameters, and the like. Following evaluation, the multi-well plates may be ejected from housing 12 and discarded.

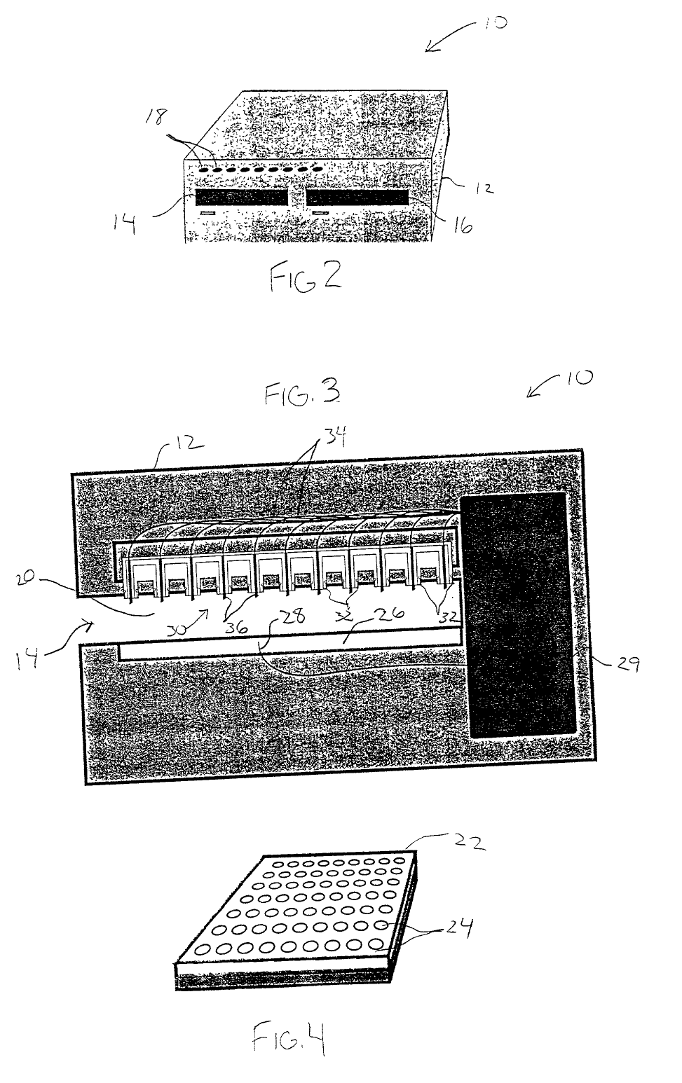

Referring now to FIG. 3, device 10 will be described schematically. Input 14 leads to a generally open interior 20 for holding a multi-well plate 22 having a plurality of wells 24 (see FIG. 4). Although not shown, it will be appreciated that a similar interior is in communication with input 16. When plate 22 is positioned within interior 20, it is held over a chamber 26 having a common electrode 28. In use, chamber 26 is filled with an electrolyte solution so that electrical current may be provided through holes in each of wells 24 by energizing common electrode 28 as described hereinafter. Common electrode 28 is coupled to a control unit 29 having the appropriate electronics to provide current to common electrode 28.

Disposed above interior 20 is a multi-well dispensing device 30 having a plurality of dispensing tips 32. Coupled to each of the dispensing tips 32 is a line 34 leading to a reservoir in control unit 29. In this way, cells in solution may be supplied to each dispensing tip 32 which in turn provides the cells in solution into wells 24 of plate 22. Conveniently, each dispensing tip 32 further includes a well electrode 36 that provides a return current path from common electrode 28. Each of well electrodes 36 is coupled to the electronics within control unit 29 so that a voltage gradient may be produced across cell membranes of the cells deposited in each of the holes in wells 24. Further, control unit 29 includes the appropriate electronics to measure and record voltage and current changes for each of the cell membranes.

To capture a cell into through holes in each of wells 24, a pressure differential is provided between each well 22 and chamber 26 to force the cells into through holes. This may be accomplished by providing positive pressure through each of the dispensing tips 32 or by applying a vacuum within chamber 26. This may be controlled by control unit 29.

Control unit 29 further includes appropriate electronics to record and store the electrophysiological measurements. Control unit 29 may include appropriate input and output ports to permit this data to be electronically transferred to another computer or other storage device for future use.

Further, control unit 29 may be employed to lower dispensing tips 32 into wells 24 after plate 22 has been inserted into input 14. Following lowering of dispensing tips 32, control unit 29 may then be employed to dispense the cells into solution into each of wells 24 as previously described. Once the operation is complete, control unit 29 may be employed to automatically eject plate 22 from input 14 so that it may be removed and discarded.

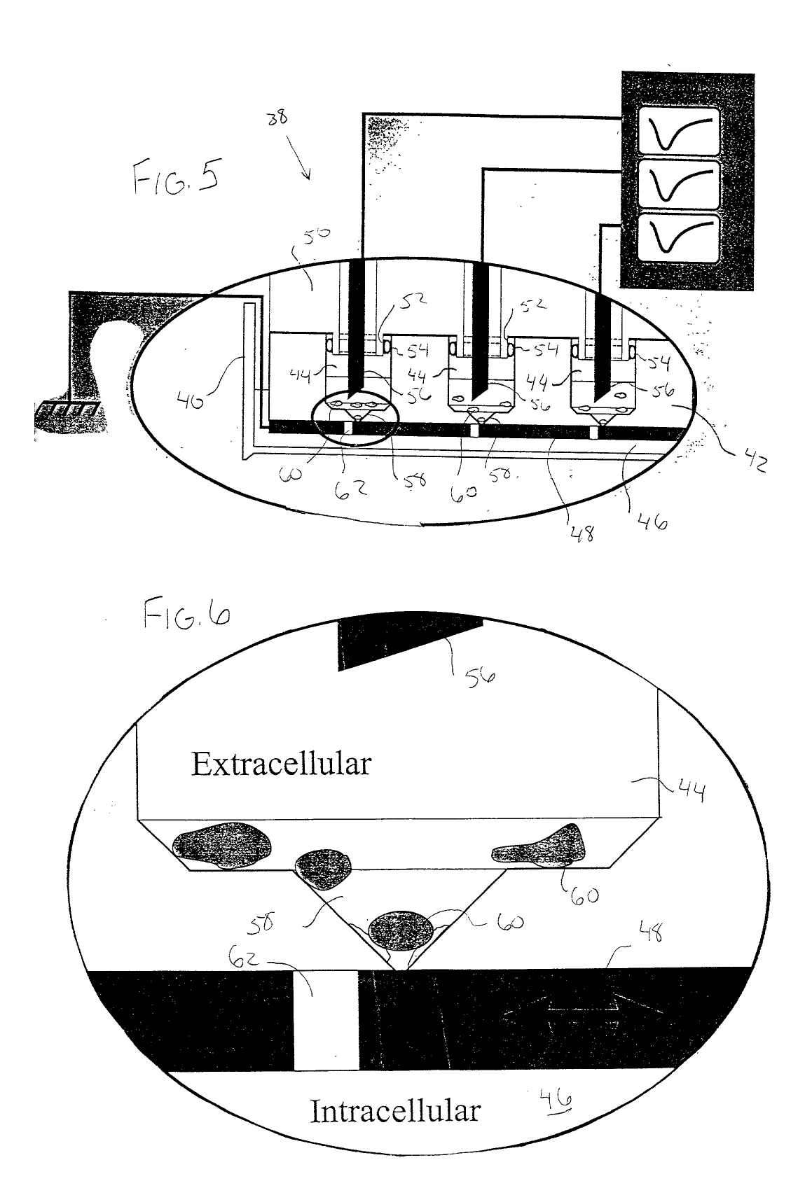

Referring now to FIG. 5, another embodiment of an electrophysiological measuring device 38 will be described. Device 38 comprises a housing 40 having an interior for holding a multi-well plate 42 having a plurality of wells 44. For convenience of illustration, only three wells are shown. However, it will be appreciated that device 38 can be constructed to have a wide variety of well configurations. Further, plate 42 need not be horizontal, but could be positioned at other orientations. Disposed below plate 42 is a chamber 46 for holding an electrolyte solution. Reciprocatably disposed within chamber 46 is a common electrode 48 that is constructed of a metal plate. Electrode 48 is coupled to appropriate electronics (not shown) to permit a voltage gradient to be applied across cell membranes as described hereinafter.

Disposed above plate 42 is a multi-well dispensing device 50 having a plurality of dispensing tips 52. Dispensing device 50 is configured so that dispensing tips 52 may be inserted into wells 44 after plate 42 is inserted into device 38. Conveniently, dispensing tips 52 may include a seal 54 to provide a seal between dispensing tips 52 and wells 44 when a pressure differential is applied to wells 44 as described hereinafter.

Conveniently, each dispensing tip 52 further includes a well electrode 56. In this way, a voltage gradient may be provided between common electrode 48 and well electrodes 56 when performing electrophysiological measurements of cells. Electrodes 56 are further coupled to appropriate electronics so that voltage and current measurements may be taken and recorded as illustrated in FIG. 5.

The end of each well 44 includes a tapered through hole 58 to provide a path for electrical current between common electrode 48 and well electrodes 56. With such a configuration, cells 60 may be dispensed into wells 44 using dispensing device 50. Cells 60 are preferably dispensed in a solution that is electrically conductive. Chamber 46 may also be filled with an electrically conductive solution so that a voltage gradient may be applied across the cell membranes of the cells in each well 44.

FIGS. 6-9 illustrate one method for utilizing device 38 to take and record electrophysiological parameters of the cell membranes. As best shown in FIG. 6, cells 60 in a solution are dispensed into each well 44 using dispensing device 38. Common electrode 48 includes a plurality of openings 62 to correspond with each through hole 58. Initially, common electrode 48 may be shifted so that openings 62 are offset from through hole 58. In this way, the solution in wells 44 will not migrate into chamber 46.

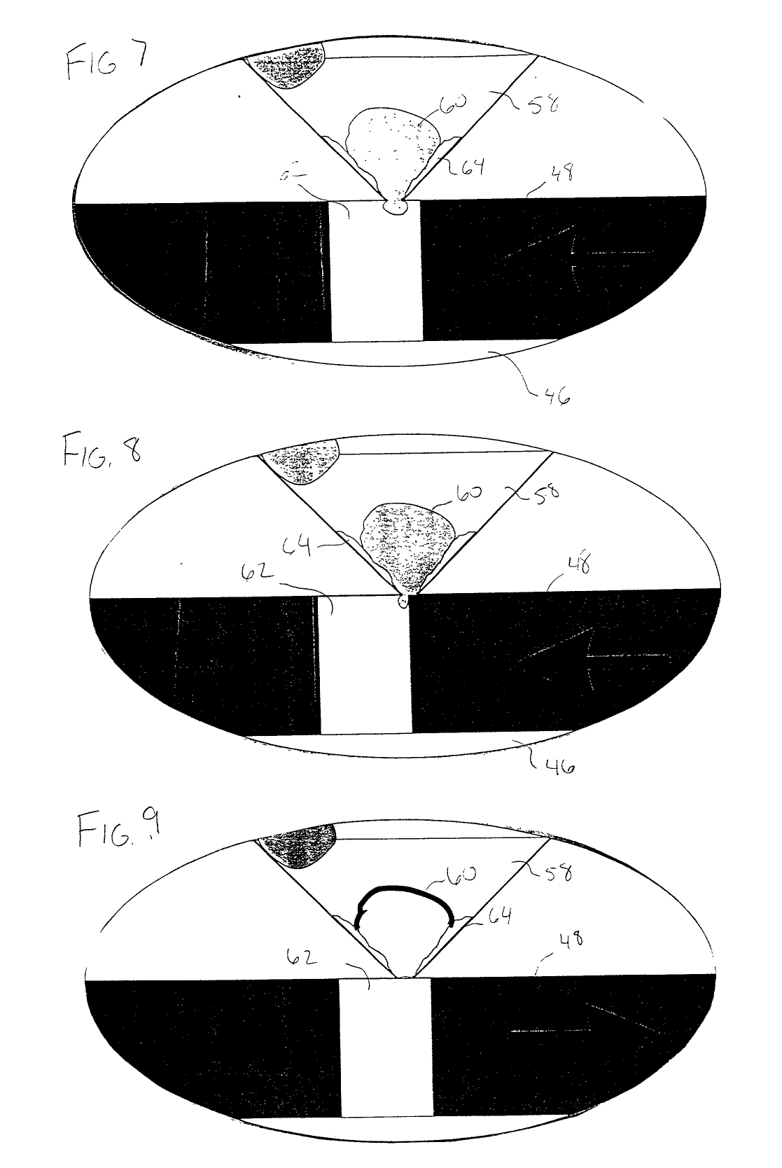

As shown in FIG. 7, electrode 48 is translated to align opening 62 with through hole 58. This causes the solution in wells 44 to flow into chamber 46. Further, a pressure differential may be provided to draw one of the cells 60 to the end of through hole 58 as shown. Such a pressure differential may be provided by supplying positive pressure through dispensing tips 52 and/or by providing a vacuum within chamber 46. The amount of pressure may be varied depending on the type of seal to be created between cell 60 and the side of through hole 58. For example, the side of through hole 58 may optionally include a glue-like substance to create a high resistance seal between cell 60 and the side wall of through hole 58. Such a glue is illustrated by reference numeral 64 in the figures. If glue 64 is not employed, an appropriate pressure differential may be provided to provide a Giga-seal between cell 60 and the side wall of through hole 58. Optionally, a potential difference may be provided by applying a voltage difference between the electrodes to determine if an appropriate seal has been created. If not, the wells with a “bad” seal will be excluded from consideration.

As shown in the optional step of FIG. 8, electrode 48 may be translated to perforate a bottom portion of cell 60 that extends below through hole 58. In this way, the interior part of cell 60 may be placed at the same potential as common electrode 48 when electrode 48 is moved back to the home position and a voltage gradient is applied as illustrated in FIG. 9. As an alternative to using electrode 48 as a cutter, device 38 may utilize large pressure pulses to destroy the bottom portion of cell 60 or may use a Nystatin solution to create holes in the bottom portion of cell 60.

In the position shown in FIG. 9, electrophysiological measurements may be made by applying a voltage gradient and measuring the current flowing through the ion channels in the cell membrane. Hence, by utilizing device 38, multiple cells may be evaluated in parallel in a high throughput manner. Once the measurements are made, plate 42 may be removed and discarded.

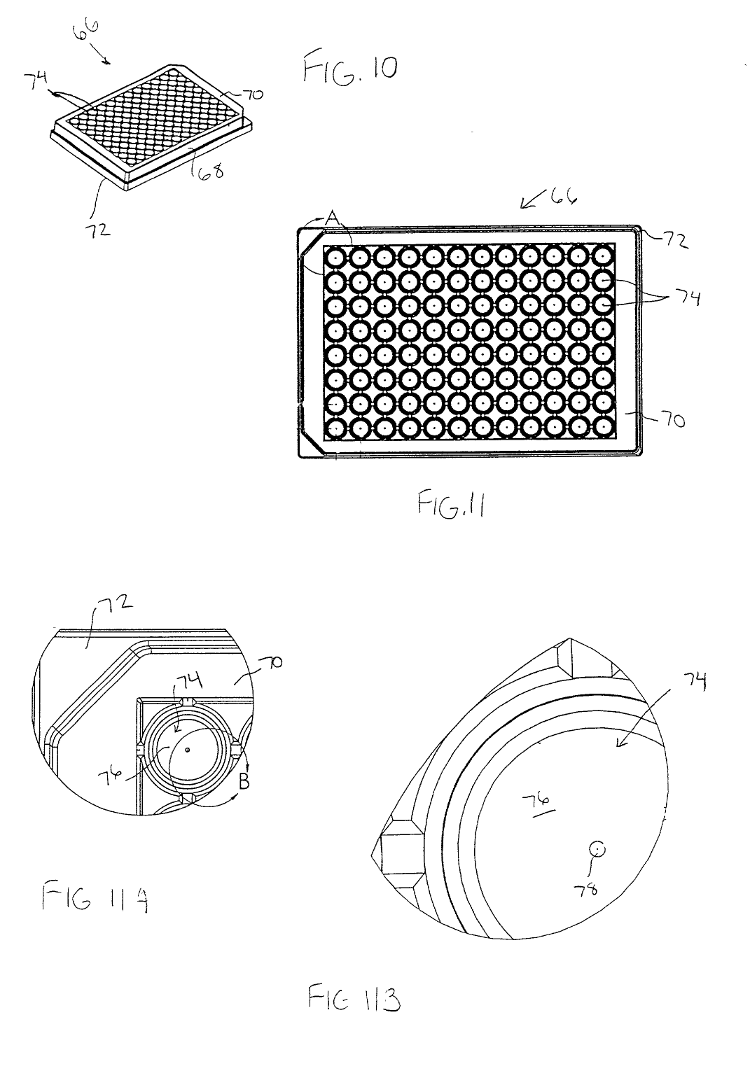

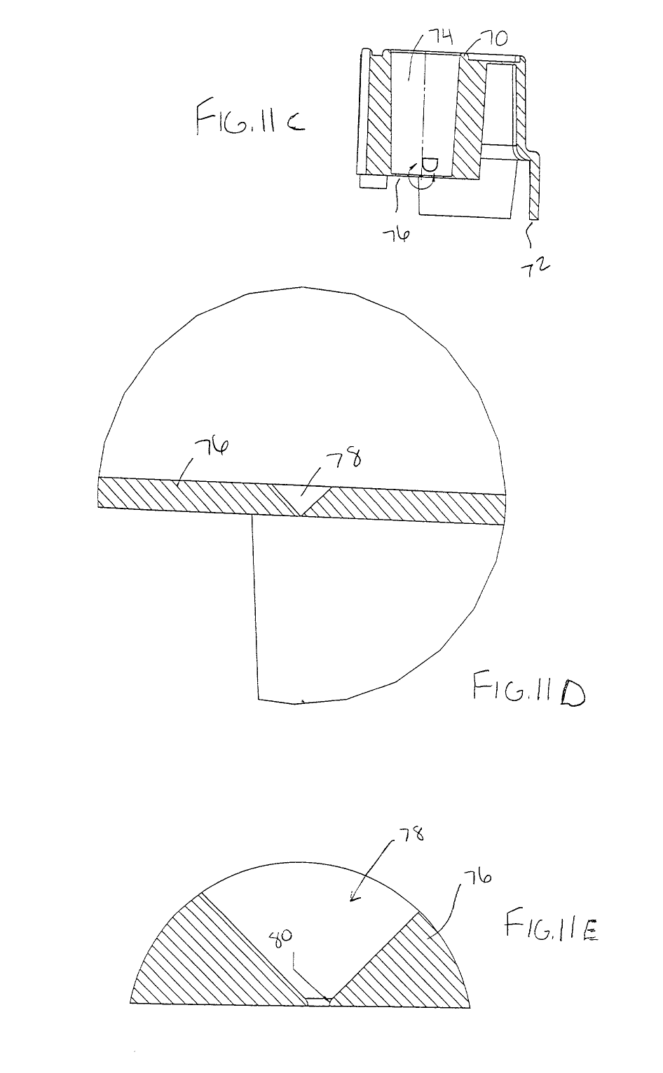

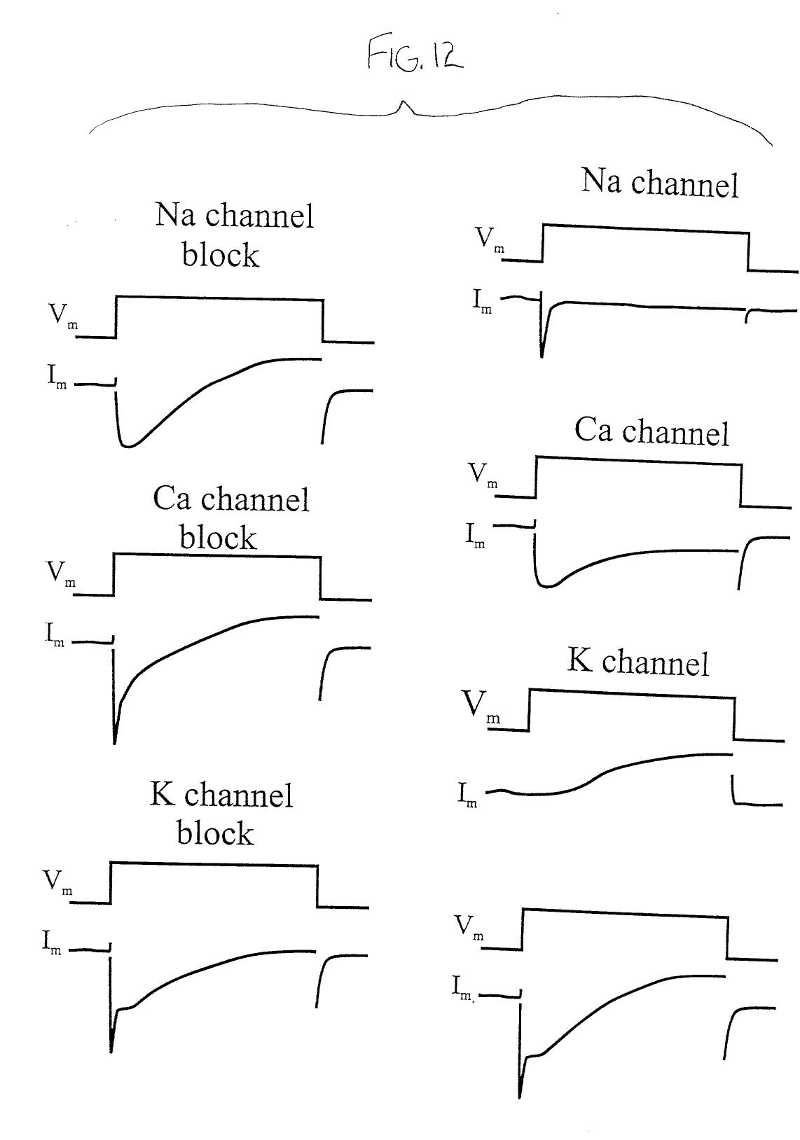

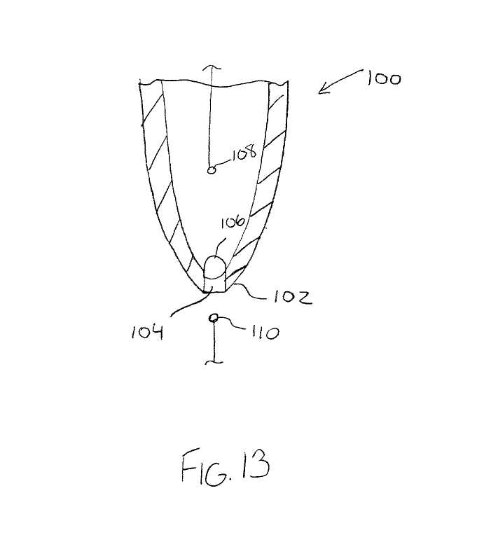

Referring now to FIGS. 10 and 11, one embodiment of a multi-well plate 66 that may be used with any of the measuring devices of the invention will be described. Plate 66 is constructed of a plate body 68 having an end 70 and an end 72. A plurality of wells are formed in the plate body, with the well being open at end 70. Further, each of wells 74 has a end 76. Conveniently, plate body may be constructed of plastic, with end 76 being constructed of glass. For example, a glass sheet may be bonded to the bottom of a polystyrene 96 well plate. In this way, plate 66 is relatively inexpensive to manufacture and may be discarded after use. Formed in each well end 76 is a through hole 78. Each through hole 78 is generally conical in geometry and tapers inward toward end 72, although the hole may taper in the opposite direction was well. Conveniently, a rounded end 80 may be provided in through hole 78 as best illustrated in FIG. 11E. One exemplary technique for forming through hole 78 is by using a laser drilling process. Such a process is able to form the conical opening, with rounded end 80 having a diameter that is approximately 2 microns to about 5 microns.

Multi-well plate 66 may be used in any of the evaluation devices described herein. In use, a pressure differential may be provided to force a cell into through hole 78 where it forms a high resistance seal with the wall of through hole 78 as previously described. Optionally, a glue-like substance may be placed into through hole 78 to facilitate the creation of a high resistance seal as previously described.

FIG. 12 illustrates voltage and current measurements across a cell membrane that were obtained using multi-well plate 66. FIG. 12 illustrates the various voltage and currents that are separated out by different ion channels.

Although shown as a flat ended well with an electrode on each side of the dielectric material having the hole, it will be appreciated that other well configurations may be used. For example, the well may be constructed of essentially any dielectric material having a hole that is smaller than the cell being tested. A pair of electrodes may then be placed on either side of the dielectric material. One such example of a well 100 is illustrated in FIG. 13. Well 100 is configured as a tube having a pointed end 102 having through hole 104 that is smaller than a cell 106 that is captured at end 102 to form an appropriate seal. Electrodes 108 and 110 are positioned on opposite sides of the cell 106 to permit measurements to be taken as previously described. Optionally, electrode 108 may be integrally formed in the wall of well 100, or could be constructed of a thin film metal electrode that is adjacent the wall of well 100.

The invention has now been described in detail for purposes of clarity of understanding. However, it will be appreciated that certain changes and modifications may be practiced within the scope of the appended claims.

What is claimed is:

1. A device to facilitate electrophysiological measurements of a biological material, the device comprising:
at least one well having an end and a side wall in the end that defines an opening;
a glue disposed on the side wall of the opening that is adapted to create a high resistance seal between a cell and the side wall;
a first electrode that is positionable in the well; and
a second electrode that is positionable outside the well to permit a voltage gradient to be produced across a membrane of a cell that is positioned within the opening, whereby electrophysiological measurements of the cell membrane may be recorded.

2. A device as in claim 1, wherein the glue is configured to create a high resistance seal having a leakage resistance of about 600 mega-ohms to about 1.1 giga-ohms.

3. A device as in claim 1, wherein the glue comprises a silicone base glue.

4. A device as in claim 1, further comprising a plate having the well along with a plurality of other wells that each have a side wall defining an opening in an end, a chamber adjacent the plate, the chamber being in fluid communication with each of the holes and being adapted to hold an electrically conductive solution, and further comprising a set of first electrodes that are positionable within each of the other wells, and wherein the second electrode comprises a common electrode that is disposed in the chamber.

5. A device to facilitate electrophysiological measurements of a biological material, the device comprising:
a plate having a plurality of wells that each have an end, wherein at least some of the wells have a hole formed in the end, wherein the holes are configured to receive an individual cell such that a high resistance seal is formed between the cell and the end;
a chamber adjacent the plate, the chamber being in fluid communication with each of the holes and being adapted to hold an electrically conductive solution;
a common electrode;
a plurality of well electrodes that are configured to be positioned within the wells to create a voltage gradient across cell membranes of the cells that are positioned within the holes so that electrophysiological measurements of the cells may be taken.

6. A device as in claim 5, wherein each hole is tapered.

7. A device as in claim 6, wherein the narrowest dimension of each hole is in the range from about 1 μm to about 5 μm.

8. A device as in claim 5, wherein each hole includes a glue that is adapted to form a seal between walls of the hole and the cell.

9. A device as in claim 5, further comprising a multi-channel liquid dispensing system having a plurality of dispensers that are configured to place the cells in solution into the wells.

10. A device as in claim 9, wherein the well electrodes are coupled to the dispensers.

11. A device as in claim 5, further comprising a vacuum source coupled to the chamber to produce a vacuum within the chamber.

12. A device as in claim 9, further wherein each dispenser includes a seal member to form a seal with the well such that positive pressure may be supplied to each well.

13. A device as in claim 9, further comprising electronics to measure voltage and/or current values for each of the wells.

14. A device as in claim 13, further comprising a controller to control operation of the liquid dispensing system and the electronics.

15. A device as in claim 5, further comprising a voltage source coupled to the common electrode.

16. A device as in claim 5, further comprising means to create small holes in the cells.

17. A device as in claim 16, wherein the hole forming means is selected from a group consisting of the common electrode when reciprocated, a pressurized solution and a hole forming solution.

18. A device to facilitate the evaluation of ion channels of a biological material, the device comprising:
a support means having means for holding cells, wherein the holding means each includes a hole that is configured to receive an individual cell such that a high resistance seal is formed between the cell holding means;
a means for storing an electrically conductive fluid disposed below the support means;
means for creating a voltage gradient across cell membranes in each of the holes; and
means for taking and recording voltage and/or current measurements of the cells.

19. A device as in claim 18, further comprising glue means for creating a high resistance seal between the cells and the holding means.

20. A method for evaluating electrical currents flowing through ion channels of a cell, the method comprising:
providing at least one well having an end and a side wall in the end that defines an opening;
placing a glue on the side wall of the opening;
depositing a cell into the opening where the glue creates a high resistance seal between the cell and the side wall;
creating a potential difference across the cell membrane and recording voltage and/or current measurements.

21. A method as in claim 20, further comprising providing a plurality of wells having side walls that define openings, placing the glue on each of the side walls, depositing a cell into each well, and creating the potential differences across each cell membrane and recording voltage and/or current measurements.

22. A method for evaluating electrical currents flowing through ion channels of a plurality of cells, the method comprising:
providing a plate having a plurality of wells that each have an end, wherein at least some of the wells have a hole formed in the end, a chamber that is disposed adjacent the plate and that is filled with an electrolyte solution, and a common electrode;
dispensing cells in a solution into the wells;
applying a pressure differential between the wells and the chamber to collect cells into the holes and create a high resistance seal between the cells and the ends of the wells;
producing a potential difference between the common electrode and well electrodes that are positioned within the wells and taking electrophysiological measurements of the cells positioned within the holes.

23. A method as in claim 22, further comprising testing whether an appropriate seal has been created between the cells and the ends of the wells.

24. A method as in claim 22, wherein the high resistance seal is at least about one giga-ohm.

25. A method as in claim 22, further comprising placing a glue into the holes to create the seal between the cells and the ends of the wells.

26. A method as in claim 25, wherein the glue produces a high resistive seal of about 600 mega-ohms to about 1.1 giga-ohms.

27. A method as in claim 22, further comprising simultaneously dispensing the cells into the wells, and simultaneously taking the voltage and current measurements.
